SEQUENCE LISTING (7 sequences)

SEQ ID NO: 1 - DNA, 20 bp, synthetic construct
  source 1..20: mol_type = other DNA
ggctaaatcgctccaccaag

SEQ ID NO: 2 - DNA, 59 bp, synthetic construct
  misc_feature 59: note = NH2-modified
  source 1..59: mol_type = other DNA
ggctaaatcgctccaccaaggatataaggagtggataccgatgaaggatataaggagtg

SEQ ID NO: 3 - DNA, 39 bp, synthetic construct
  source 1..39: mol_type = other DNA
caacaatcaggacgataccgatgaaggatataaggagtg

SEQ ID NO: 4 - DNA, 39 bp, synthetic construct
  source 1..39: mol_type = other DNA
gtcctgattgttgcttcatcggtatccactccttatatc

SEQ ID NO: 5 - DNA, 20 bp, synthetic construct
  source 1..20: mol_type = other DNA
ggctaaatcgctccaccaag

SEQ ID NO: 6 - DNA, 10 bp, synthetic construct
  source 1..10: mol_type = other DNA
cgatttagcc

SEQ ID NO: 7 - DNA, 49 bp, synthetic construct
  source 1..49: mol_type = other DNA
  misc_feature 49: note = NH2-modified
caacaatcaggacgataccgatgaagcaacaatcaggaccttggtggag

(19) United States
(12) Patent Application Publication
(10) Pub. No.: US 2025/0305031 A1
(43) Pub. Date: Oct. 2, 2025

(54) ULTRASENSITIVE METHOD FOR MEASURING ANALYTE

(71) Applicant: SEKISUI MEDICAL CO., LTD., Tokyo (JP)

(72) Inventors: Akane OOMORI, Tokyo (JP); Tetsuo SEKINO, Tokyo (JP)

(73) Assignee: SEKISUI MEDICAL CO., LTD., Tokyo (JP)

(21) Appl. No.: 18/857,531

(22) PCT Filed: Apr. 6, 2023
(86) PCT No.: PCT/JP2023/014274

§ 371 Date: Jun. 17, 2025

(30) Foreign Application Priority Data
Apr. 20, 2022 (JP) 2022-069657

(51) Int. Cl. C12Q 1/682; C12Q 1/6804

(52) U.S. Cl. CPC C12Q 1/682; C12Q 1/6804

(57) ABSTRACT

Provided is an ultrasensitive method for measuring an anti-drug antibody that is simpler and less expensive than conventional methods. Provided is an ultrasensitive method for measuring an analyte using a capture probe and an assist probe and adopting an improved PALSAR method. By using the capture probe and the assist probe and adopting the improved PALSAR method in a double antigen bridging immunoassay, ultrasensitive measurement of an anti-drug antibody can be performed simply and inexpensively.

### TECHNICAL FIELD

The present invention relates to an ultrasensitive method for measuring a target antibody, particularly an anti-drug antibody, and a target nucleic acid, particularly nucleic acid therapeutics using an improved signal amplification method, and a kit for use in such a measurement method.

### BACKGROUND ART

As a method for amplifying a signal originating from a nucleic acid in a sample, a method using a pair of self-assembly probes composed of first and second oligonucleotides (also referred to as honeycomb probes or HCPs) (PALSAR method) is known (JP3267576 B2). Various types of research and development have already been made to improve operability of the PALSAR method, shorten reaction time, increase efficiency of the signal amplification, etc. (JP3310662 B2, JP3912595 B2, JP4121757 B2).

Furthermore, “a method for detecting a target gene” has also been devised (JP4482557 B2), where the first oligonucleotide corresponds to “a first probe composed of three nucleic acid regions, a nucleic acid region X, a nucleic acid region Y, and a nucleic acid region Z in order from 5′-end side” and the second oligonucleotide corresponds to “a second probe composed of three nucleic acid regions, a nucleic acid region X′ complementary to the nucleic acid region X, a nucleic acid region Y′ complementary to the nucleic acid region Y, and a nucleic acid region Z′ complementary to the nucleic acid region Z in order from 5′-end side,” the method using “an assist probe having a structure provided with the nucleic acid region X, the nucleic acid region Y, the nucleic acid region X, and a target region hybridizable to a target gene in order from the 5′-end side, or a structure provided with the target region, the nucleic acid region Z, the nucleic acid region Y, and the nucleic acid region Z in order from the 5′ end side.” Further, a method of designing an assist probe suitable for the PALSAR method has also been developed (JP5289314 B2).

The procedure of the PALSAR method in these conventional art is outlined as follows:
1. A capture probe is immobilized in a strip well type 96-well microplate, a sample containing a target oligo DNA and an assist probe solution are added thereto and allowed to react for a certain period of time, followed by washing with washing solution;
2. After the washing, the washing solution is thoroughly drained from the 96-well microplate, first and second oligonucleotides labeled with digoxigenin are added thereto and allowed to react for a certain period of time, followed by washing with washing solution;
3. After washing the microplate well, an alkaline phosphatase-labeled anti-digoxigenin antibody is added thereto and allowed to react in an incubator at 37° C.;
4. After washing with washing solution, a luminescent substrate solution of alkaline phosphatase is added and allowed to react in a dark place for a certain period of time, and thereafter luminescence intensity (RLU) is measured using a luminometer.

The PALSAR method using honeycomb probes and an assist probe is highly versatile, excellent in specificity and quantitativity, and highly sensitive, so that it is used for detection of oligonucleotides such as nucleic acid therapeutics which are difficult to amplify by PCR (JP6718032 B2, JP6995250 B2).

Meanwhile, a double antigen bridging immunoassay using a capture drug antibody and a tracer drug antibody is known as a method for measuring an anti-drug antibody (JP4902674 B2). The method of JP4902674 B2 is characterized in that the capture drug antibody is a mixture of said drug antibody comprising at least two of said drug antibodies that differ in the antibody site at which they are conjugated to the solid phase and have the same amino acid sequence, and the tracer drug antibody is a mixture of said drug antibody comprising at least two of said drug antibodies that differ in the antibody site at which they are conjugated to the detectable label and have the same amino acid sequence.

However, in the method of JP4902674 B2, as described above, it is necessary to prepare two or more drug antibodies that differ in the antibody site at which they are bound to the solid phase or the detectable label and have the same amino acid sequence, respectively for the capture drug antibody and the tracer drug antibody, i.e., the total of four or more drug antibodies should be prepared. Therefore, a high cost and a long time are required for the preparation of the both antibodies. Also, the performance management of immunoassay using said drug antibodies and the quality control of reagents become complicated.

Furthermore, in the conventional PALSAR method (JP6718032 B2, JP6995250 B2), it is necessary to prepare honeycomb probes for each measurement, resulting in complicated operation and wasteful disposal of an unused, excessive solution.

Therefore, there is a need for a simpler and less expensive method for measuring an analyte such as a target antibody and a target nucleic acid.

The present inventors found that ultrasensitive measurement of an analyte is enabled simply and inexpensively by using a capture nucleic acid (referred to as “capture probe” or abbreviated to “CP” in the present description) and a tracer nucleic acid (referred to as “assist probe” or abbreviated to “AP” in the present description), and by adopting an improved PALSAR method. The capture probe and the assist probe can be chemically synthesized in an inexpensive and extremely simple manner, and can also be easily modified in various ways.

Furthermore, the performance management of assay and the quality control of reagents can be performed simply.

### CITATION LIST

### Patent Literature

Patent Document 1: JP3267576 B2
Patent Document 2: JP3310662 B2
Patent Document 3: JP3912595 B2
Patent Document 4: JP4121757 B2
Patent Document 5: JP4482557 B2
Patent Document 6: JP5289314 B2
Patent Document 7: JP6718032 B2
Patent Document 8: JP6995250 B2
Patent Document 9: JP4902674 B2

### SUMMARY OF INVENTION

### Technical Problem

The problem to be solved by the present invention is to provide an ultrasensitive method for measuring an analyte that is simpler and less expensive than conventional methods.

### Solution to Problem

For solving the problem by the present invention, an ultrasensitive method for measuring an analyte using a capture probe and an assist probe as well as an improved PALSAR method is provided. That is, the present invention is constituted of the following configurations <Embodiment 1> to <Embodiment 19>.

### Embodiment 1

A method for detecting a target antibody in a sample, the method comprising:
(i) providing an assembly in which a pair of self-assembly probes composed of first and second oligonucleotides and an assist probe are hybridized to each other (hereinafter, also referred to as a “signal probe polymer”);
(ii) bringing the sample containing the target antibody into contact with an epitope for the target antibody contained in a capture probe to form a [capture probe]-[target antibody] complex in a liquid phase different from that of the step (i);
(iii) bringing the signal probe polymer into contact with the [capture probe]-[target antibody] complex to form a [capture probe]-[target antibody]-[signal probe polymer] complex; and
(iv) detecting the [capture probe]-[target antibody]-[signal probe polymer]complex.

### Embodiment 2

A method for detecting a target antibody in a sample, the method comprising:
(i) providing an assembly in which a pair of self-assembly probes composed of first and second oligonucleotides and an assist probe are hybridized to each other;
(ii) bringing the sample containing the target antibody and a capture probe into contact with the assembly to form a complex of the capture probe, the target antibody, the assist probe, and a plurality of first and second oligonucleotides;
(iii) removing a liquid phase from a solid phase that is bound to the capture probe or washing the solid phase, thereby removing the first and second oligonucleotides that are not involved in formation of the complex; and
(iv) detecting a label contained in the first or second oligonucleotide.

### Embodiment 3

A method for quantifying a target antibody in a sample, the method comprising:
(i) providing an assembly in which a pair of self-assembly probes composed of first and second oligonucleotides and an assist probe are hybridized to each other;
(ii) bringing the sample and a capture probe into contact with the assembly;
(iii) removing a liquid phase from a solid phase or washing the solid phase, wherein the solid phase is bound to the capture probe; and
(iv) quantifying a signal from a label contained in the first or second oligonucleotide.

### Embodiment 4

The method according to embodiment 1, comprising separating and removing the target antibody which is in a state free from the [capture probe]-[target antibody] complex without being involved in formation of the complex.

### Embodiment 5

The method according to any one of embodiments 1 to 4, wherein the capture probe is immobilized on a solid phase before the capture probe is brought into contact with the target antibody.

### Embodiment 6

The method according to any one of embodiments 1 to 5, wherein the first and second oligonucleotides are labeled with a ruthenium complex, peroxidase, fluorescent dye, biotin, or digoxigenin.

### Embodiment 7

The method according to any one of embodiments 1 to 6, wherein the target antibody is a monospecific antibody that binds to one antigen or a bispecific antibody (diabody).

### Embodiment 8

The method according to any one of embodiments 1 to 7, wherein the target antibody is an anti-drug antibody.

### Embodiment 9

The method according to any one of embodiments 1 to 8, wherein the sample is derived from a biological sample.

### Embodiment 10

An assembly formed by bringing a pair of self-assembly probes composed of first and second oligonucleotides into contact with an assist probe.

### Embodiment 11

A kit for detecting a target antibody in a sample, comprising:
(1) a capture probe;
(2) an assist probe; and
(3) a pair of self-assembly probes composed of first and second oligonucleotides,
wherein the capture probe and the assist probe each contains a nucleic acid and has an epitope to which the target antibody binds.

### Embodiment 12

A kit for detecting a target antibody in a sample, comprising:
(1) a capture probe; and
(2) an assembly of a pair of self-assembly probes composed of first and second oligonucleotides and an assist probe,
wherein the capture probe and the assist probe each contains a nucleic acid and has an epitope to which the target antibody binds.

### Embodiment 13

The assembly or the kit according to any one of embodiments 10 to 12, wherein the first and second oligonucleotides are labeled with a ruthenium complex, peroxidase, fluorescent dye, biotin, or digoxigenin.

### Embodiment 14

The kit according to any one of embodiments 11 to 13, wherein the target antibody is a monospecific antibody that binds to one antigen or a bispecific antibody (diabody).

### Embodiment 15

The kit according to any one of embodiments 11 to 14, wherein the target antibody is an anti-drug antibody.

### Embodiment 16

The kit according to any one of embodiments 11 to 15, wherein the sample is derived from a biological sample.

### Embodiment 17

The kit according to any one of embodiments 11 to 16, wherein the epitope is a nucleic acid, polypeptide, carbohydrate chain, protein, polymer compound, middle molecular compound, low molecular compound, or a part thereof.

### Embodiment 18

The kit according to any one of embodiments 11 to 16, wherein the epitope is 5-methylated cytosine, phosphorothioate nucleic acid, boranophosphate nucleic acid, morpholino nucleic acid, LNA, BNA, 2′-O-methylated RNA (2′-OMe), 2′-O-methoxyethylated RNA (2′-MOE), 2′-F-RNA, ENA® (2′-O,4′-C-Ethylene-bridged Nucleic Acids), N-acetyl galactosamine (GalNAc) nucleic acid, or polyethylene glycol.

### Embodiment 19

The method according to any one of embodiments 1 to 9, wherein the capture probe and the assist probe each contains a nucleic acid and has an epitope to which the target antibody binds.

### Advantageous Effects of Invention

By using the capture probe and the assist probe and adopting the improved PALSAR method in the double antigen bridging immunoassay, it is possible to perform ultrasensitive measurement of an analyte simply and inexpensively.

### BRIEF DESCRIPTION OF DRAWINGS

FIG. 1 is a diagram showing an aspect of a basic process of the present invention.

FIG. 2 is a graph showing improvement in detection sensitivity and an S/N ratio of a target antibody by the method of the present invention as compared with the conventional method. The signal is represented by a bar graph and the S/N ratio is represented by a line graph.

FIG. 3 is a graph showing quantitativity of the method of the present invention.

FIG. 4 is a graph showing measurement results of the conventional method where a capture probe is immobilized on a solid phase in advance.

FIG. 5 is a graph showing measurement results obtained by the method of the present invention where a capture probe is immobilized on a solid phase in advance.

FIG. 6 is a graph showing improvement in detection sensitivity and an S/N ratio of a target nucleic acid by the method of the present invention as compared with the conventional method. The signal is represented by a bar graph and the S/N ratio is represented by a line graph.

### DESCRIPTION OF EMBODIMENTS

A substance to be measured (analyte) by the measurement method of the present invention is an antibody (target antibody) contained in a sample, particularly an anti-drug antibody, and a nucleic acid (target nucleic acid), particularly nucleic acid therapeutics. Hereinafter, a clinically important anti-drug antibody and nucleic acid therapeutics are described by way of examples, but a person skilled in the art will understand that the method of the present invention is not limited thereto.

### I. Ultrasensitive Method for Measuring Analyte

In the present description, unless otherwise specified, the terms “measurement method” and “detection method” are used in their broadest senses including the identical concept. Therefore, the method of the present invention can be used as a measurement method, detection method, quantitative measurement method, or qualitative measurement method of an analyte such as a target antibody and a target nucleic acid, by measuring the strength of detected signals.

The term “target antibody” herein means an antibody to be measured. The term “anti-drug antibody” herein as one example of the target antibody means an antibody directed to a drug. Such an antibody may possibly be produced, for example, as an immunogenic reaction in a patient receiving a drug during drug therapy. The “anti-drug antibody” to be measured is not particularly limited as long as it is contained in biological samples. Preferred is IgG, IgM, IgD, IgE, or IgA, and more preferred is IgG or IgM.

Examples of the above anti-drug antibody include an “anti-nucleic acid therapeutics antibody”. Examples of the “nucleic acid therapeutics” in the term “anti-nucleic acid therapeutics antibody” include: siRNA; miRNA; antisense; aptamer; decoy; ribozyme; CpG oligo; and others (PolyI:PolyC (double-stranded RNA) for the purpose of activating innate immunity, antigene, or the like), which are known in the art. Moreover, the “nucleic acid therapeutics” also includes: a gene transfer vector for use in genetic medicine; a gene contained in a genetic vaccine; and further a medicine containing a nucleic acid chain as an active ingredient such as a polydeoxyribonucleotide compound including defibrotide sodium (CAS registration number: 83712-60-1). Additionally, the “nucleic acid therapeutics” means an oligonucleotide composed of two or more nucleotides, where a nucleic acid constituting the oligonucleotide may have a non-natural structure (so-called a nucleic acid analogue) as well as a natural structure. However, the nucleic acid analogue such as 5-FU (5-fluorouracil) itself is not included in the “nucleic acid therapeutics” in the present invention. The “nucleic acid therapeutics” of the present invention may be a single-stranded nucleic acid or a double-stranded nucleic acid. Further, in the case of a double-stranded nucleic acid, it may be a hetero double-stranded nucleic acid. Note that the term “nucleic acid” herein means a polymer of nucleotide, but may also refer to a nucleotide itself depending on the context.

When an anti-drug antibody is produced against the above “nucleic acid therapeutics,” an epitope to which the anti-drug antibody binds in the nucleic acid therapeutics can be a base moiety, a sugar moiety, or a phosphate moiety of a specific nucleotide in the oligonucleotide. Further, the number of nucleic acids constituting the epitope to which the anti-drug antibody binds can be either a specific nucleotide alone, or a nucleic acid composed of two or more nucleotides (oligonucleotide). Furthermore, if the oligonucleotide is modified to make it a nucleic acid drug, such as to modulate the strength of complementary binding, biodegradation resistance, or DDS, the epitope to which the anti-drug antibody binds may be the chemical moiety of such modification. Such modification includes: modification of sugars and phosphates in nucleotides as described below; cyclic and hairpin structures of the oligonucleotide; molecules other than nucleic acids in the oligonucleotide sequence; steric structures formed by molecules other than said nucleic acids with the oligonucleotide; and addition of polyethylene glycols.

The term “target nucleic acid” herein means a nucleic acid to be measured. See above for the “nucleic acid therapeutics” as one example of the target nucleic acid. The term “target nucleic acid” herein may refer to either DNA or RNA which may be single-stranded or double-stranded, or may be chemically modified as long as it can form a specific hybrid with a capture probe and an assist probe. Examples of the chemical modification include phosphorothioate modification (sulfurization), 2′-F modification, 2′-O-Methyl (2′-OMe) modification, 2′-O-Methoxyethyl (2′-MOE) modification, morpholino modification, LNA modification, BNACOC modification, BNANC modification, ENA modification, and cEt BNA modification. When the above target nucleic acid is double-stranded, it is made single-stranded and used in the present invention. Preferably, the target nucleic acid is, but not limited to, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, or 30-mer nucleotide length.

A “sample” used in the detection method of the present invention is not particularly limited as long as it is a biologically derived component and an analyte can be present therein. The sample is preferably the whole blood, serum, plasma, lymph fluid, or saliva of a human, monkey, dog, swine, rat, guinea pig, or mouse, and particularly preferably a blood-derived component such as the whole blood, serum, or plasma of a human. These may be used after dilution with water or a buffer solution. Further, the sample of the present invention also includes those obtained by diluting an analyte at a known concentration with water, a buffer solution, or a biologically derived component containing no analyte (for example, a blood-derived component) to adjust the concentration.

The above-described sample may be subjected to a pretreatment as necessary. For example, the property of the analyte in the sample is changed by mixing with an acid or a surfactant, or substances affecting formation of a [capture probe]-[analyte]-[assist probe] complex in the sample is separated and removed by filtration through a filter capable of sieving a specific molecular weight fraction.

Generally used buffer solution may be used as the above buffer solution, and examples thereof include: tris-hydrochloric acid, boric acid, phosphoric acid, acetic acid, citric acid, succinic acid, phthalic acid, glutaric acid, maleic acid, glycine, and salts thereof; and Good's buffer solutions such as MES, Bis-Tris, ADA, PIPES, ACES, MOPSO, BES, MOPS, TES, and HEPES. Examples of the water include RNase- and DNase-free water. Note that the water used in the preparation of the buffer solution, or the like, is also preferably RNase- or DNase-free water.

The “capture probe” and “assist probe” used in the detection method of the present invention are not particularly limited as long as each contains a nucleic acid and can be chemically synthesized, and are preferably DNA (deoxyribonucleic acid), RNA (ribonucleic acid), PNA (peptide nucleic acid), or a chemically modified nucleic acid. They can also include non-natural nucleotide or any modification group.

Any site of a base moiety, a sugar moiety, or a phosphate moiety of the nucleic acid may be targeted for chemical modification. Examples of the above chemically modified nucleic acid include phosphorothioate modification (sulfurization), 2′-F modification, 2′-O-Methyl (2′-OMe) modification, 2′-O-Methoxyethyl (2′-MOE) modification, morpholino modification, LNA modification, BNA^{COC} modification, BNA^{NC} modification, ENA modification, and cEtBNA modification.

Among these, preferred is a locked nucleic acid (LNA), bridged nucleic acid (BNA), phosphorothioate oligonucleotide, morpholino oligonucleotide, boranophosphate oligonucleotide, 2′-O-methylated RNA (2′-OMe), 2′-O-methoxyethylated RNA (2′-MOE), or 2′-F-RNA.

The above nucleic acid may be either single-stranded or double-stranded.

The “capture probe” and “assist probe” used in the present invention each contains at least a moiety to which the analyte binds (analyte-binding-moiety) in the structure, and each may further contain an optional sequence. The sequence of the “capture probe” and the sequence of the “assist probe” may be identical to or different from each other. Furthermore, when the analyte is an anti-drug antibody, the above-described “nucleic acid therapeutics” itself can be used as the “capture probe” and “assist probe” used in the detection method of the present invention.

Specific structures of the “capture probe” in the present invention include, but are not limited to, the following examples:
5′-(n)a-(analyte-binding-moiety)-(n)b-(functional group)-3′, wherein “n” represents any nucleotide, “a” and “b” each independently represents 0 or a natural number, provided that the requirements of the nucleic acid chain length described later are satisfied, “analyte-binding-moiety” represents a moiety to which an analyte binds, and “functional group” represents a functional group such as an amino group which modifies the capture probe;
5′-(n)a-(analyte-binding-moiety)-(n)b-(adaptor)-3′, wherein “n” represents any nucleotide, “a” and “b” each independently represents 0 or a natural number, provided that the requirements of the nucleic acid chain length described later are satisfied, “analyte-binding-moiety” represents a moiety to which an analyte binds, and “adaptor” represents an adaptor which binds to the capture probe, such as biotin, streptavidin, or avidin, and a combination thereof, antigen or antibody, and a combination thereof, the same applies hereinafter;
5′-(functional group)-(n)a-(analyte-binding-moiety)-(n)b-3′, wherein “n” represents any nucleotide, “a” and “b” each independently represents 0 or a natural number, provided that the requirements of the nucleic acid chain length described later are satisfied, “analyte-binding-moiety” represents a moiety to which an analyte binds, and “functional group” represents a functional group such as an amino group which modifies the capture probe; and
5′-(adaptor)-(n)a-(analyte-binding-moiety)-(n)b-3′, wherein “n” represents any nucleotide, “a” and “b” each independently represents 0 or a natural number, provided that the requirements of the nucleic acid chain length described later are satisfied, “analyte-binding-moiety” represents a moiety to which an analyte binds, and “adaptor” represents an adaptor such as biotin which binds to the capture probe.

Specific structures of the “assist probe” in the present invention include, but are not limited to, the following examples:
5′-(n)c-(analyte-binding-moiety)-(n)d-(tag sequence)-3′, wherein “n” represents any nucleotide, “c” and “d” each independently represents 0 or a natural number, provided that the requirements of the nucleic acid chain length described later are satisfied, “analyte-binding-moiety” represents a moiety to which an analyte binds, and “tag sequence” represents a tag sequence which is partially the same as one of sequences in a pair of self-assembly probes described later (the tag sequence is partially complementary to the other sequence in the pair of self-assembly probes); and
5′-(tag sequence)-(n)c-(analyte-binding-moiety)-(n)d-3′, wherein “n” represents any nucleotide, “c” and “d” each independently represents 0 or a natural number, provided that the requirements of the nucleic acid chain length described later are satisfied, “analyte-binding-moiety” represents a moiety to which an analyte binds, and “tag sequence” represents a tag sequence which is partially the same as one of sequences in a pair of self-assembly probes described later (the tag sequence is partially complementary to the other sequence in the pair of self-assembly probes).

In one aspect, where the first oligonucleotide has a constitution of an oligonucleotide composed of a nucleic acid region X, a nucleic acid region Y, and a nucleic acid region Z in order from the 5′ end side, and the second oligonucleotide has a constitution of an oligonucleotide composed of a nucleic acid region X′ complementary to the nucleic acid region X, a nucleic acid region Y′ complementary to the nucleic acid region Y, and a nucleic acid region Z′ complementary to the nucleic acid region Z in order from the 5′ end side,
the tag sequence has a constitution of:
an oligonucleotide composed of the nucleic acid region X of the first oligonucleotide, the nucleic acid region Y of the first oligonucleotide, and the nucleic acid region X of the first oligonucleotide in order from the 5′ end side; or an oligonucleotide composed of the nucleic acid region Z of the first oligonucleotide, the nucleic acid region Y of the first oligonucleotide, and the nucleic acid region Z of the first oligonucleotide in order from the 5′ end side; or
an oligonucleotide composed of the nucleic acid region X′ of the second oligonucleotide, the nucleic acid region Y′ of the first oligonucleotide, and the nucleic acid region X′ of the first oligonucleotide in order from the 5′ end side; or an oligonucleotide composed of the nucleic acid region Z′ of the second oligonucleotide, the nucleic acid region Y′ of the second oligonucleotide, and the nucleic acid region Z′ of the second oligonucleotide in order from the 5′ end side.

When the analyte is an anti-drug antibody, an epitope in the “capture probe” and “assist probe” used in the detection method of the present invention, to which the anti-drug antibody binds, can be identified, for example, by a method using an affinity sensor based on surface plasmon resonance (SPR) as a detection principle, or an antigen competition method. The “capture probe” and “assist probe” of the present invention preferably have identical epitopes to which the anti-drug antibody binds.

When the analyte is the target nucleic acid, the “capture probe” used in the present invention is a probe for capturing the target nucleic acid and contains a nucleic acid probe and a solid phase flanking a 3′-end or 5′-end nucleotide of the nucleic acid probe.

When the analyte is the target nucleic acid, the “assist probe” used in the present invention is a probe for detecting the target nucleic acid and contains a nucleic acid probe and a tag or label flanking a 5′-end or 3′-end nucleotide of the nucleic acid probe.

In the present invention, “capturing” the target nucleic acid by the capture probe primarily means that the nucleic acid probe contained in the capture probe is hybridized to the target nucleic acid. In one aspect, “capturing” the target nucleic acid by the capture probe means that the target nucleic acid indirectly binds to the solid phase contained in the capture probe via the nucleic acid probe contained in the capture probe.

In the present description, hybridization of the nucleic acid probe contained in the capture probe or the assist probe with the target nucleic acid means that the nucleic acid probe binds to the target nucleic acid having a specific nucleotide sequence through base pairing to form a double-stranded nucleic acid molecule, the nucleic acid probe having a sequence complementary to a part of the specific nucleotide sequence.

The capture probe is one or two or more species, and may be a combination of two or more species having different sites for binding to a solid phase. Also, the assist probe is one or two or more species, and may be a combination of two or more species.

The “capture probe” used in the detection method of the present invention may contain an adaptor for binding to the solid phase described later, and the “assist probe” may include chemical modification for binding the label for detecting an analyte described later.

Examples of the “adaptor” used in the present invention include: biotin, streptavidin, or avidin, and a combination thereof; and antigen or antibody, and a combination thereof. Preferred are biotin, streptavidin, or avidin, and a combination thereof.

Nucleic acid chain length of the “capture probe” and “assist probe” in the present invention is not particularly limited. The nucleic acid chain length suitable for the detection method of the present invention can be appropriately designed by taking into consideration desired specificity, sensitivity, and the like in detection of an analyte. In one aspect, the nucleic acid probe contained in the capture probe is 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, or 25-mer nucleotide length. In another aspect, the nucleic acid probe contained in the capture probe is 5-mer, 6-mer, 7-mer, 8-mer, 9-mer, 10-mer, 11-mer, 12-mer, 13-mer, 14-mer, 15-mer, or 16-mer nucleotide length. In yet another aspect, the nucleic acid probe contained in the capture probe is 5-mer, 6-mer, 7-mer, 8-mer, 9-mer, 10-mer, or 11-mer nucleotide length. A preferred example is a case that the chain length of the capture probe and the assist probe are the same, or a case that the nucleic acid chain length of the assist probe is longer than that of the capture probe by 1-mer to 60-mers. More preferred examples include cases that the nucleic acid chain length of the assist probe is longer than that of the capture probe by 5-mers to 55-mers, 10-mers to 50-mers, 15-mers to 45-mers, 20-mers to 40-mers, 25-mers to 35-mers, 25-mers to 40-mers, 25-mers to 45-mers, 25-mers to 50-mers, 30-mers to 40-mers, 30-mers to 45-mers, 30-mers to 50-mers, 35-mers to 45-mers, and 35-mers to 50-mers. In one aspect, the nucleic acid probe contained in the assist probe is 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, 50-mer, 51-mer, 52-mer, 53-mer, 54-mer, 55-mer, 56-mer, 57-mer, 58-mer, 59-mer, 60-mer, 61-mer, 62-mer, 63-mer, 64-mer, 65-mer, 66-mer, 67-mer, 68-mer, 69-mer, 70-mer, 71-mer, 72-mer, 73-mer, 74-mer, 75-mer, 76-mer, 77-mer, 78-mer, 79-mer, 80-mer, 81-mer, 82-mer, 83-mer, 84-mer, or 85-mer nucleotide length. In another aspect, the nucleic acid probe contained in the assist probe is 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, 40-mer, 41-mer, 42-mer, 43-mer, 44-mer, 45-mer, 46-mer, 47-mer, 48-mer, 49-mer, 50-mer, 51-mer, 52-mer, 53-mer, 54-mer, 55-mer, 56-mer, 57-mer, 58-mer, 59-mer, or 60-mer nucleotide length. In yet another aspect, the nucleic acid probe contained in the assist probe is 4-mer, 5-mer, 6-mer, 7-mer, 8-mer, 9-mer, 10-mer, 11-mer, 12-mer, 13-mer, 14-mer, 15-mer, 16-mer, 17-mer, 18-mer, 19-mer, 20-mer, 21-mer, 22-mer, 23-mer, 24-mer, 25-mer, 26-mer, 27-mer, 28-mer, 29-mer, 30-mer, 31-mer, 32-mer, 33-mer, 34-mer, 35-mer, 36-mer, 37-mer, 38-mer, 39-mer, or 40-mer nucleotide length.

When the analyte is an anti-drug antibody, the above nucleic acid chain has a sequence derived from nucleic acid therapeutics containing an epitope to which an anti-nucleic acid therapeutics antibody binds, and may further have an optional sequence not having an identical or analogous structure to the epitope, wherein the nucleic acid therapeutics is the cause of the anti-nucleic acid therapeutics antibody production. When the nucleic acid chain length of the assist probe is longer than the nucleic acid chain length of the capture probe, said longer portion of the nucleic acid chain length is preferably an optional sequence not having an identical or analogous structure to the epitope.

The term “bring into contact” or “contact step” herein means to place one substance and the other substance adjacently to each other to form a chemical bond such as a covalent bond, ionic bond, metal bond, or non-covalent bond between the substances. In one aspect of the present invention, the “contact” step of a sample with the capture probe and the assist probe in the detection method of the present invention is performed by mixing a liquid sample, a solution containing the capture probe, and a solution containing the assist probe in any combination.

When the analyte is an anti-drug antibody, the “epitope to which an anti-drug antibody binds” contained in the capture probe and the assist probe used in the detection method of the present invention is as described above. The epitope is not particularly limited as long as the anti-drug antibody can bind to, and is preferably a nucleic acid, polypeptide, carbohydrate chain, protein, polymer compound, middle molecular compound, low molecular compound, or a part thereof.

Examples of the “epitope to which an anti-drug antibody binds” include, but are not limited to, the following;
5-methylated cytosine, phosphorothioate nucleic acid, boranophosphate nucleic acid, morpholino nucleic acid, LNA, BNA, 2-O-methylated RNA (2′-OMe), 2′-O-methoxyethylated RNA (2′-MOE), 2′-F-RNA, ENA® (2′-O,4′-C-Ethylene-bridged Nucleic Acids), N-acetyl galactosamine (GalNAc) nucleic acid, and polyethylene glycol.

The term “self-assembly/self-aggregation” herein means a state that a plurality of first oligonucleotides hybridizes to the second oligonucleotide to form a complex, and a state that a plurality of second oligonucleotides hybridizes to the first oligonucleotide to form a complex.

The “pair of self-assembly probes” used in the detection method of the present invention is composed of the first and second oligonucleotides. When simply referred to as “first oligonucleotide” and “second oligonucleotide” in the present description, they mean the first oligonucleotide and the second oligonucleotide constituting the pair of self-assembly probes, respectively. The first oligonucleotide and the second oligonucleotide have complementary nucleotide sequence regions which are hybridizable to each other, enabling to form an oligonucleotide polymer by a self-assembly reaction. Preferably, at least one of the first and second oligonucleotides is labeled with a labeling substance. Herein, in one aspect, “hybridizable” means the state of being completely complementary in said complementary nucleotide sequence regions. Further, in another aspect, “hybridizable” means the state of being complementary except for one or two mismatches in said complementary nucleotide sequence regions.

It is also possible to label the pair of self-assembly probes with a labeling substance for detection in advance. Preferred examples of such a labeling substance include a radioactive isotope, biotin, digoxigenin, fluorescent substance, luminescent substance, pigment, or metal complex.

The labeling substance is preferably a ruthenium complex, biotin, or digoxigenin, and the oligonucleotide labeling is preferably performed by labeling the 5′ end or the 3′ end.

The “pair of self-assembly probes” is more specifically described as oligonucleotides includes: the first oligonucleotide containing at least a nucleic acid region X, a nucleic acid region Y, and a nucleic acid region Z in order from the 5′ end side; and the second oligonucleotide containing at least a nucleic acid region X′ complementary to the nucleic acid region X, a nucleic acid region Y′ complementary to the nucleic acid region Y, and a nucleic acid region Z′ complementary to the nucleic acid region Z in order from the 5′ end side. Because the self-assembled form is associated with a honeycomb, one or both of this pair of self-assembly probes may be referred to as a honeycomb probe(s) (HCP(s)).

In a case that the capture probe is bound to a solid phase, examples of the “solid phase” include insoluble microparticles, microbeads, fluorescent microparticles, magnetic particles, a microplate, a microarray, a microscope slide, and a substrate such as an electrically conductive substrate.

Examples of the binding of the capture probe to the solid phase include a chemical bonding method, a biological interaction method, and a physical adsorption method. In the chemical bonding method, for example, when using a solid phase coated with a functional group such as a carboxyl group, it is possible to modify the capture probe with a functional group such as an amino group in advance, and then cause a coupling reaction with the functional group. For example, in the biological interaction method, the avidity between streptavidin coated on a solid phase and biotin pre-bound to the capture probe can be exploited. Furthermore, in the physical adsorption method, for example, when using a solid phase having a negative electrical charge, it is possible to electrostatically adsorb the capture probe to the solid phase by labeling the capture probe with a substance having a positive electrical charge such as an amino group.

Examples of the functional group for modifying the capture probe include, but are not limited to, the following:
an amino group, a carboxyl group, a thiol group, and a maleimide group.

The method of “washing” the solid phase to which the capture probe binds is not particularly limited. For example, a given amount of washing solution is added to the solid phase, and left to stand still or gently shaken to separate and remove the solution in the solid phase. Examples of the separation and removal methods of the solution preferably include decantation, centrifugal separation, and aspiration.

The step of separating and removing the solution by “decantation” is generally performed by tilting the solid phase to remove the solution.

The step of separating and removing the solution by “centrifugal separation” is generally performed by centrifugation at 500 to 3000×g for 0.2 to 5 minutes at 20 to 30° C., centrifugation at 800 to 1500×g for 0.5 to 2 minutes at 23 to 28° C., and preferably centrifugation at 1000×g for 1 minute at 25° C. to generate a supernatant, and then removing the supernatant.

The step of separating and removing the solution by “aspiration” is generally performed by using a micropipette or an aspirator. More specifically, the micropipette or aspirator may be used in accordance with the manufacturer's instruction manual.

After a [capture probe]-[analyte]-[assist probe]-[honeycomb probes] complex bound to the solid phase is formed, decantation, centrifugal separation, or aspiration is preferably used for “separating and removing” the honeycomb probes, which is in a free state without being involved in the formation of said complex, from the [capture probe]-[analyte]-[assist probe]-[honeycomb probes] complex. Specific separation and removal methods are the same as the methods of separating and removing the solution. Examples of methods for detecting a label contained in the honeycomb probes include a turbidity method, absorbance method, fluorophotometry, electrochemiluminescence method, and flow cytometry. Preferred is an electrochemiluminescence method.

The electrochemiluminescence method is performed by, for example, applying electric energy to an electrically conductive substrate to which the [capture probe]-[analyte]-[assist probe]-[honeycomb probes] complex binds, and detecting light emitted by reduction of a ruthenium complex which has been labeled to the honeycomb probes in advance.

In one aspect of the present invention, a kit for detecting an analyte in a sample includes at least the following components:
(1) a capture probe;
(2) an assist probe; and
(3) a pair of self-assembly probes composed of first and second oligonucleotides,
wherein the capture probe and the assist probe each contains a nucleic acid and has a moiety to which the analyte binds.

In another aspect, a kit for detecting an analyte in a sample includes at least the following components:
(1) a capture probe; and
(2) an assembly of a pair of self-assembly probes composed of first and second oligonucleotides and an assist probe,
wherein the capture probe and the assist probe each contains a nucleic acid and has a moiety to which the analyte binds.

Each component is as described above.

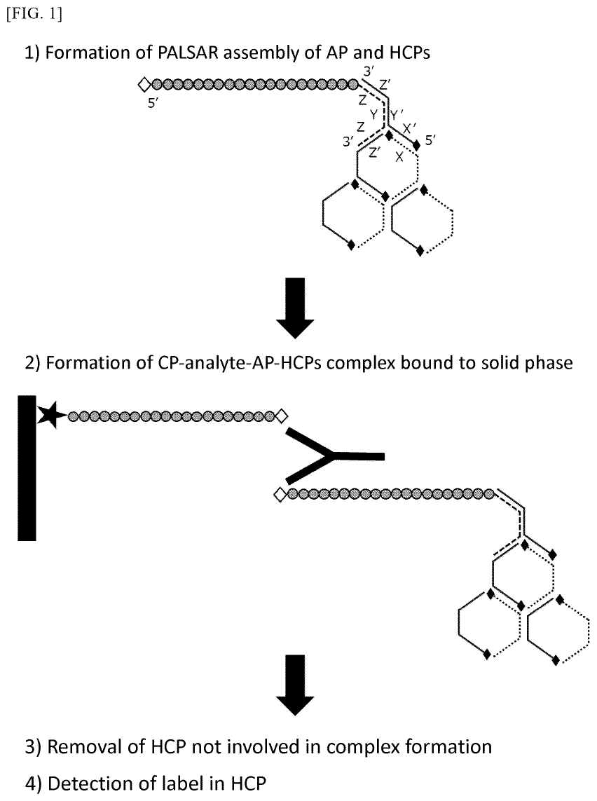

One aspect of the basic process of the present invention will be described with reference to FIG. 1.

Firstly, an assembly of the assist probe and the honeycomb probes (AP-HCPs assembly) is provided. This AP-HCPs assembly can be formed by sequential or simultaneous contact during measurement. In the pair of self-assembly probes composed of first and second oligonucleotides (honeycomb probes), the first oligonucleotide contains the nucleic acid region X, the nucleic acid region Y, and the nucleic acid region Z in order from the 5′ end side, and the second oligonucleotide contains the nucleic acid region X′, the nucleic acid region Y′, and the nucleic acid region Z′ in order from the 5′ end side. X and X′, Y and Y′, and Z and Z′ are complementary to each other (hereinafter, said nucleic acid regions may be simply referred to as X, Y, Z, X′, Y′, and Z′). In addition, in the aspect illustrated in FIG. 1, the 5′-end of the first and second oligonucleotides is labeled with digoxigenin (black diamond shapes in FIG. 1). Y′ and Z′ of the second oligonucleotide hybridize to Y and Z of the assist probe, and X′, Y′, and Z′ of the second oligonucleotide hybridize to X, Y, and Z of the first oligonucleotide, respectively. Then, X and X′, Y and Y′, and Z and Z′ are hybridized repeatedly one after another to form a signal probe polymer of self-assembly probes. An assembly formed and stored in advance before performing the measurement may be provided as this AP-HCPs assembly.

This AP-HCPs assembly is sequentially or simultaneously brought into contact with the capture probe and a sample containing an analyte to be measured (anti-drug antibody in the figure) to form a CP-analyte-AP-HCPs complex. The capture probe and the assist probe each contains a moiety to which the analyte binds (white diamond shapes in FIG. 1). The capture probe may be bound to a solid phase before, during, or after the formation of the complex.

Thereafter, the solid phase is washed or a liquid phase is removed to remove HCP which is not involved in the formation of the complex, and a label contained in HCP is detected.

In one embodiment, a sample containing the [capture probe]-[analyte]-[assist probe]-[honeycomb probes] complex is brought into contact with a ruthenium complex-labeled anti-digoxigenin antibody. By detecting light emitted from the ruthenium complex, it is possible to measure a concentration of the analyte, and the like.

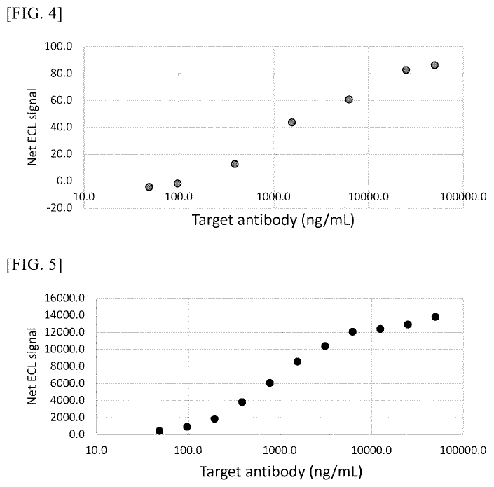

Specific basic examples of the PALSAR method are shown in FIGS. 4 to 14 of WO 2013/172305, etc. They may be modified as appropriate for the application to the self-assembly reaction of the present invention based on common general knowledge of a person skilled in the art, by using a probe for dimer formation or the like.

The method of the present invention has extremely high specificity because the [capture probe]-[analyte]-[assist probe] complex is formed via moieties in the capture probe and the assist probe, to which the analyte binds. Further, when the analyte is an anti-drug antibody, the Ig class of the anti-drug antibody involved in formation of the complex is not limited. Thereby, when anti-drug antibodies of the IgG, IgM, IgD, IgE, or IgA class are present in the sample, two or more Ig classes can be detected by one measurement. Herein, “one measurement” means that the contact of the sample, the capture probe, and the assist probe is once. With this feature, the detection method of the present invention can sensitively detect the presence of the anti-drug antibody regardless of dosage interval or dosage period of a drug (nucleic acid therapeutics), or sample collection time, without an influence of Ig class switching.

Accordingly, the present invention can also be used for data acquisition to determine dosage regimen of nucleic acid therapeutics in an individual receiving the nucleic acid therapeutics, confirmation of the possibility of production of an anti-nucleic acid therapeutics antibody in development of the nucleic acid therapeutics, and design of nucleic acid therapeutics itself by specifying an epitope to which said anti-drug antibody binds. Furthermore, the measurement method of the present invention can be used for highly sensitive measurement of a nucleic acid therapeutics concentration in a biological sample of an animal or a human to which the nucleic acid therapeutics is administered; in pharmacokinetic/pharmacodynamic (PK/PD) screening tests in an exploratory stage of drug development; in safety tests, pharmacological tests, and pharmacokinetic tests in a nonclinical stage; and in a clinical stage.

Hereinafter, specific aspects will be described in Examples for more easily understanding the present invention. However, the present invention is not limited to these Examples.

### EXAMPLES

### [Example 1] Comparison Between Conventional Method and Method of Present Invention

### 1. Experimental Material and Method

### [Conventional Method]

### (1) Target Antibody

As a measurement target, an anti-digoxigenin antibody (available from MBL Co., Ltd., product number M227-3) was subjected to the test after preparation to 300 ng/mL with an antibody diluent. Additionally, a blank sample of an antibody diluent containing no target antibody (0 ng/mL) was also measured at the same time.

### (1-1) Composition of Antibody Diluent

One hundred and thirty-seven (137) mM Sodium Chloride, 8.1 mM Disodium Phosphate, 2.68 mM Potassium Chloride, 1.47 mM Potassium Dihydrogenphosphate, 0.02% Tween 20, 1.5 ppm ProClin 300 (hereinafter, 1×PBS-TP), and 1% BSA (bovine serum albumin)

### (2) Capture Probe

As a capture probe, CP-ADA-5Dig-GTI2040-3Biotin was used, which was obtained by labeling the 3′ end and the 5′ end of the sequence of GTI-2040 developed as nucleic acid therapeutics with biotin and digoxigenin, respectively. The nucleobase sequence thereof is 5′-GGCTAAATCGCTCCACCAAG-3′, and synthesis thereof was requested to INTEGRATED DNA TECHNOLOGIES, Inc. (HPLC purification grade). Furthermore, CP-ADA-5Dig-GTI2040-3Biotin was subjected to the test after preparation to 1 pmol/μL with Nuclease-Free Water.

### (3) Assist Probe

As an assist probe, a tracer nucleic acid (AP-ADA-5Dig-GTI2040-ZYZ) was used, which is composed of the same nucleotide sequence as the capture probe (20 nucleotides) and the nucleotide sequence partially the same as a probe for signal amplification (HCP-1), and is labeled with digoxigenin at the 5′ end. Synthesis thereof was requested to INTEGRATED DNA TECHNOLOGIES, Inc. (HPLC purification grade).

<Sequence of AP-ADA-5Dig-GTI2040-ZYZ>
5′- GGCTAAATCGCTCCACCAAG
GATATAAGGAGTGGATACCGATGAAGGATATAAGGAGTG-(NH2)-3′.

The tracer nucleic acid was subjected to the test after preparation to 1 pmol/μL with Nuclease-Free Water.

### (4) Formation of [Target Antibody]-[Capture Probe]-[Assist Probe] Complex (Antibody Bridging Reaction)

To a 96-well round bottom plate, 300 ng/mL of the target antibody and 0 ng/mL of the blank sample were each dispensed in aliquots of 50 μL, and 50 μL of a bridging reaction solution was further added, respectively to obtain a 100 μL mixture in total. The mixture was allowed to react at 37° C. for 3 hours while stirring at 700 rpm.

### (4-1) Composition of Bridging Reaction Solution

To 40.3 μL of 1×PBS-TP, 0.8 μL of 1 pmol/μL CP-ADA-5Dig-GTI2040-3Biotin and 0.8 μL of 1 pmol/μL AP-ADA-5Dig-GTI2040-ZYZ were added.

### (5) Blocking of Measurement Plate

One hundred and fifty (150) μL of a blocking solution was added to a streptavidin-immobilized measurement plate (Meso Scale Diagnostics, LLC., product number L45SA-1), and allowed to react at 37° C. for 1 hour while stirring at 700 rpm. Then, the plate was washed twice with 200 μL of 1×PBS-TP.

### (5-1) Composition of Blocking Solution

3% BSA-added 1×PBS-TP

### (6) Immobilization of [Target Antibody]-[Capture Probe]-[Assist Probe] Complex to Measurement Plate

After the bridging reaction, 75 μL of the reaction solution was added to the measurement plate after blocking, and allowed to react at 37° C. for 1 hour while stirring at 700 rpm. Then, the plate was washed twice with 200 μL of 1×PBS-TP.

### (7) Detection Assisting Reaction

Fifty (50) μL of a detection assisting reaction solution was added to the measurement plate after immobilization of the [target antibody]-[capture probe]-[assist probe] complex, and allowed to react at 37° C. for 1 hour while stirring at 700 rpm. Then, the plate was washed twice with 200 μL of 1×PBS-TP.

### (7-1) Composition of Detection Assisting Reaction Solution

To 147 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 7.0 μL of 50×Denhardt's Solution, 70 μL of 5% PEG 8000, 2.4 μL of 10 pmol/μL Ru complex-labeled HCP-1, 3.0 μL of 10 pmol/μL Ru complex-labeled HCP-2, and 119.0 μL of Nuclease-Free Water were added.

### (7-2) Nucleotide Sequence of HCP-1

The nucleic acid probe (HCP-1) used in Example 1 includes a sequence complementary to a nucleotide sequence of HCP-2 in the pair of self-assembly probes, and is labeled with a Ru complex at the 5′ end.

<Nucleotide sequence of HCP-1>
5′- CAACAATCAGGACGATACCGATGAAGGATATAAGGAGTG -3′

### (7-3) Nucleotide Sequence of HCP-2

The nucleic acid probe (HCP-2) used in Example 1 includes a sequence partially complementary to a nucleotide sequence of AP-ADA-M-DNA1-ZYZ-3N in the pair of self-assembly probes, and is labeled with a Ru complex at the 5′ end.

<Nucleotide sequence of HCP-2>
5′- GTCCTGATTGTTGCTTCATCGGTATCCACTCCTTATATC -3′

### (8) Detection

To the measurement plate after the detection assisting reaction, 150 μL of 0.5×Read Buffer [two-fold dilution of [MSD GOLD Read Buffer A (Meso Scale Diagnostics, LLC., product number R92TG-1)] with sterile purified water] was added, and light emission amount from the Ru-labeled HCP-1 and HCP-2 was measured using a Meso QuickPlex SQ 120 MM system (manufactured by Meso Scale Diagnostics, LLC.) to detect the target antibody.

### [Method of Present Invention]

### (1) Formation of AP-HCPs Assembly

To a DNA LoBind Tube, 108 μL of a detection assisting reaction solution with the following composition was dispensed and allowed to react at 40° C. for 1 hour while stirring at 800 rpm to form an AP-HCPs assembly.

### (1-1) Composition of Detection Assisting Reaction Solution

To 46 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 2.2 μL of 50×Denhardt's Solution, 22 μL of 5% PEG 8000, 1.1 μL of 1 pmol/μL AP-ADA-5Dig-GTI2040-ZYZ, 3.0 μL of 10 pmol/μL Ru complex-labeled HCP-1, 3.8 μL of 10 pmol/μL Ru complex-labeled HCP-2, and 31.0 μL of Nuclease-Free Water were added.

### (2) Formation of [Target Antibody]-[Capture Probe]-[AP-HCPs Assembly] Complex (Antibody Bridging Reaction)

To a 96-well round bottom plate, 300 ng/mL of the target antibody and 0 ng/mL of the blank sample were each dispensed in aliquots of 50 μL, and 50 μL of a bridging reaction solution was further added, respectively to obtain a 100 μL mixture in total. The mixture was allowed to react at 37° C. for 3 hours while stirring at 700 rpm.

### (2-1) Composition of Bridging Reaction Solution

To 323 μL of 1×PBS-TP, 0.8 μL of 1 pmol/μL CP-ADA-5Dig-GTI2040-3Biotin and 81 μL of the above AP-HCPs assembly were added.

### (3) Blocking of Measurement Plate

It was performed in the same manner as described in [Conventional method].

### (4) Immobilization of [Target Antibody]-[Capture Probe]-[AP-HCPs Assembly] Complex to Measurement Plate

After the antibody bridging reaction, 75 μL of the reaction solution was added to the measurement plate after blocking, and allowed to react at 37° C. for 1 hour while stirring at 700 rpm. Then, the plate was washed twice with 200 μL of 1×PBS-TP.

### (5) Detection

Signal detection was performed in the same manner as described in [Conventional method] using the washed measurement plate.

### 2. Result

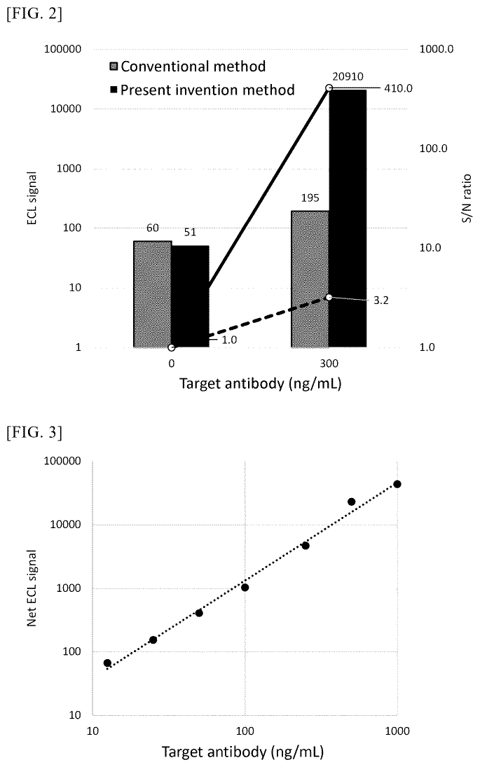

The measurement results are shown in FIG. 2. When 300 ng/mL of the target antibody was measured, the method of the present invention achieved improvement in the net signal obtained by subtracting the signal of 0 ng/mL of the blank sample by about 155 times (20859/135), and the S/N ratio by about 128 times (410/3.2) as compared with the conventional method.

### [Example 2] Quantitativity of Method of Present Invention

### 1. Experimental Material and Method

### (1) Formation of AP-HCPs Assembly

One hundred and eight (108) μL of a detection assisting solution with the following composition was dispensed to a DNA LoBind Tube and allowed to react at 40° C. for 2 hours while stirring at 800 rpm to form an AP-HCPs assembly.

### (1-1) Composition of Detection Assisting Solution

To 142 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 7.0 μL of 50×Denhardt's Solution, 67 μL of 5% PEG 8000, 6.7 μL of 1 pmol/μL AP-ADA-5Dig-GTI2040-ZYZ, 9.4 μL of 10 pmol/μL Ru complex-labeled HCP-1, 11.8 μL of 10 pmol/μL Ru complex-labeled HCP-2, and 92.0 μL of Nuclease-Free Water were added.

### (2) Target Antibody

The anti-digoxigenin antibody described in Example 1 was diluted and prepared with normal human serum to 1000, 500, 250, 100, 50, 25, and 12.5 ng/mL, and then subjected to the test. Additionally, a blank sample of normal human serum containing no target antibody (0 ng/mL) was also measured at the same time.

### (3) Formation of [Target Antibody]-[Capture Probe]-[AP-HCPs Assembly] Complex (Antibody Bridging Reaction)

To a 96-well round bottom plate, the above target antibody and the above blank sample were each dispensed in aliquots of 50 μL, and 50 μL of a bridging reaction solution was further added, respectively to obtain a 100 μL mixture in total. The mixture was allowed to react at 25° C. for 2.5 hours while stirring at 700 rpm.

### (3-1) Composition of Bridging Reaction Solution

To 1146 μL of 1×PBS-TP, 5.8 μL of 1 pmol/μL CP-ADA-5Dig-GTI2040-3Biotin and 288 μL of the above AP-HCPs assembly were added.

### (4) Blocking of Measurement Plate

It was performed in the same manner as described in [Conventional method] in Example 1.

### (5) Immobilization of [Target Antibody]-[Capture Probe]-[AP-HCPs Assembly] Complex to Measurement Plate

After the antibody bridging reaction, 75 μL of the reaction solution was added to the measurement plate after blocking, and allowed to react at 37° C. for 1 hour while stirring at 700 rpm. Then, the plate was washed twice with 200 μL of 1×PBS-TP.

### (6) Detection

Signal detection was performed in the same manner as described in [Conventional method] in Example 1 using the washed measurement plate.

### 2. Result

The measurement results are shown in FIG. 3. In the method of the present invention, quantitativity was confirmed in the range of 12.5 to 1000 ng/mL.

### [Example 3] Case of Immobilizing Capture Probe on Solid Phase in Advance

### 1. Experimental Material and Method

### [Conventional Method]

### (1) Immobilization of Capture Probe

The same capture probe as described in Example 1 was used after preparation to 0.03 fmol/μL with 1×PBS-TP. The capture probe was dispensed in aliquots of 50 μL to a streptavidin-immobilized measurement plate (Meso Scale Diagnostics, LLC., product number L45SA-1), and allowed to react at 25° C. for 45 minutes while stirring at 700 rpm. After the reaction, the plate was washed twice with 200 μL of 1×PBS-TP.

### (2) Blocking of Measurement Plate

One hundred and fifty (150) μL of the same blocking solution as described in Example 1 was added to the above measurement plate, and allowed to react at 25° C. for 40 minutes while stirring at 700 rpm. Then, the plate was washed twice with 200 μL of 1×PBS-TP.

### (3) Target Antibody

The anti-digoxigenin antibody described in Example 1 was serially diluted with normal human serum to 50000, 25000, 6250, 1563, 391, 97.7, and 48.8 ng/mL, and further diluted 50-fold with 1×PBS-TP (EDTA) to prepare a sample to be subjected to the test. Additionally, a blank sample of normal human serum containing no target antibody (0 ng/mL) was also measured at the same time.

### (3-1) Composition of 1×PBS-TP (EDTA)

To 7178 μL of 1×PBS-TP, 22 μL of 500 mM EDTA was added.

### (4) Formation Reaction of [Target Antibody]-[Capture Probe] Complex

Twenty-five (25) μL of the target antibody diluted and prepared as described above was added to the measurement plate after blocking, further 25 μL of a reaction solution was dispensed thereto and allowed to react at 25° C. for 1 hour while stirring at 700 rpm. Then, the plate was washed twice with 200 μL of 1×PBS-TP.

### (4-1) Composition of Reaction Solution

One hundred and thirty-seven (137) mM Sodium Chloride, 8.1 mM Disodium Phosphate, 2.68 mM Potassium Chloride, 1.47 mM Potassium Dihydrogenphosphate, 0.02% Tween 20, 1.5 ppm ProClin 300, 0.2 mg/mL ssDNA, and 1.5 mM EDTA (pH 8.0)

### (5) Detection Assisting Reaction

Fifty (50) μL of a detection assisting reaction solution was added to the measurement plate after formation of the [target antibody]-[capture probe] complex, and allowed to react at 25° C. for 1 hour while stirring at 700 rpm. Then, the plate was washed twice with 200 μL of 1×PBS-TP.

### (5-1) Composition of Detection Assisting Solution

To 30 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 3.0 μL of 50×Denhardt's Solution, 15.0 μL of 10 mg/mL ssDNA, 3.0 μL of 0.1 pmol/μL AP-ADA-5Dig-GTI2040-ZYZ, 1.5 μL of 10 pmol/μL Ru complex-labeled HCP-1, 1.5 μL of 10 pmol/μL Ru complex-labeled HCP-2, and 1446 μL of 1×PBS-TP (1% BSA) were added.

### (6) Detection

Signal detection was performed in the same manner as described in Example 1, [Conventional method], using the washed measurement plate.

### [Method of Present Invention]

### (1) Formation of AP-HCPs Assembly

Twenty-nine (29) μL of the detection assisting solution with the following composition was dispensed to a DNA LoBind Tube and allowed to react at 40° C. for 1 hour while stirring at 800 rpm to form an AP-HCPs assembly. After the reaction, the resultant was stored in the shade at 4° C.

### (1-1) Composition of Detection Assisting Solution

To 15.8 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 0.58 μL of 50×Denhardt's Solution, 5.8 μL of 1 pmol/μL AP-ADA-5Dig-GTI2040-ZYZ, 2.32 μL of 10 pmol/μL Ru complex-labeled HCP-1, 2.90 μL of 10 pmol/μL Ru complex-labeled HCP-2, and 11.6 μL of Nuclease-Free Water were added.

### (2) Immobilization of Capture Probe

The same capture probe as described in Example 1 was used after preparation to 0.03 fmol/μL with 1×PBS-TP. The capture probe was dispensed in aliquots of 50 μL to a streptavidin-immobilized measurement plate (Meso Scale Diagnostics, LLC., product number L45SA-1) and allowed to react at 25° C. for 45 minutes while stirring at 700 rpm. After the reaction, the plate was washed twice with 200 μL of 1×PBS-TP.

### (3) Blocking of Measurement Plate

It was performed in the same manner as described in [Conventional method] in the present example.

### (4) Target Antibody

The anti-digoxigenin antibody described in Example 1 was serially diluted two-fold with normal human serum to 50000 to 48.8 ng/mL, and further diluted 50-fold with the 1×PBS-TP (EDTA) to prepare a sample to be subjected to the test. Additionally, a blank sample of normal human serum containing no target antibody (0 ng/mL) was also measured at the same time.

### (5) Formation Reaction of [Target Antibody]-[Capture Probe] Complex

Twenty-five (25) μL of the target antibody diluted and prepared as described above was added to the measurement plate after blocking, and 25 μL of the reaction solution was further dispensed thereto and allowed to react at 25° C. for 1 hour while stirring at 700 rpm. Then, the plate was washed twice with 200 μL of 1×PBS-TP.

### (6) Formation Reaction of [Target Antibody]-[Capture Probe]-[AP-HCPs Assembly] Complex

The AP-HCPs assembly described in (1) was diluted 10-fold with the antibody diluent described in Example 1, [Conventional method], and further diluted 100-fold with a hybridization solution. The resulting AP-HCPs assembly was dispensed in aliquots of 50 μL to the measurement plate. Next, the resultant was allowed to react at 25° C. for 1 hour while stirring at 700 rpm, and washed twice with 200 μL of 1×PBS-TP.

### (6-1) Composition of Hybridization Solution

To 70.2 μL solution (containing 189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 7.02 μL of 50×Denhardt's Solution, 39.0 μL of 10 mg/mL ssDNA, 3744.8 μL of 1×PBS-TP (1% BSA), and 39.0 μL of the 10-fold diluted AP-HCPs assembly were added.

### (7) Detection

Signal detection was performed in the same manner as described in Example 1, [Conventional method], using the washed measurement plate.

### 2. Result

The measurement results of the conventional method are shown in FIG. 4, and the measurement results of the method of the present invention are shown in FIG. 5. When the capture probe was immobilized on the solid phase in advance, the measurement range of the conventional method was 390.6 to 50000 ng/mL.

On the other hand, quantitativity of the method of the present invention was confirmed in the range of 48.8 to 50000 ng/mL. Furthermore, when the sensitivity was compared in the measurement range of 390.6 to 50000 ng/mL, the method of the present invention achieved 157 to 304 times higher sensitivity than the conventional method in terms of the net signal obtained by subtracting the signal of 0 ng/mL of the blank sample.

### [Example 4] Comparison Between Conventional Method and Method of Present Invention

### 1. Experimental Material and Method

### [Conventional Method]

### (1) Target Nucleic Acid

GTI-2040 developed as nucleic acid therapeutics was used as a measurement target. The nucleotide sequence thereof is 5′-GGCTAAATCGCTCCACCAAG-3′, and synthesis thereof was requested to INTEGRATED DNA TECHNOLOGIES, Inc. (HPLC purification grade). Further, GTI-2040 was subjected to the test after preparation to 0.00078 to 0.2 nmol/L with a TE solution containing 20% human serum (pH 8.0, NIPPON GENE CO., LTD.). Additionally, a blank sample of the TE solution containing 20% human serum but no target nucleic acid (0 nmol/L) was also measured at the same time.

### (2) Capture Probe

Used as the capture probe was CP-GTI2040-10-3B which has a sequence complementary to the sequence of 10 nucleotides from the 5′ end of the target nucleic acid and is labeled with biotin at the 3′ end. The nucleotide sequence thereof is 5′-CGATTTAGCC-3′, and synthesis thereof was requested to NIHON GENE RESEARCH LABORATORIES Inc. (HPLC purification grade). Further, CP-GTI2040-10-3B was subjected to the test after preparation to 10 pmol/μL with Nuclease-Free Water.

### (3) Assist Probe

AP-XYX-GTI2040-10 was used as the assist probe, which has a sequence complementary to the sequence of 10 nucleotides from the 3′ end of the target nucleic acid and is composed of a nucleotide sequence partially the same as a probe for signal amplification (HCP-1). Synthesis thereof was requested to NIHON GENE RESEARCH LABORATORIES Inc. (HPLC purification grade). Further, AP-XYX-GTI2040-10 was subjected to the test after preparation to 1 pmol/μL with Nuclease-Free Water.

<Sequence of AP-XYX-GTI2040-10>
5′- CAACAATCAGGACGATACCGATGAAGCAACAATCAGGACCTTGGT
GGAG -(NH2)-3′

### (4) Blocking of Measurement Plate

One hundred and fifty (150) μL of a blocking solution was added to a streptavidin-immobilized measurement plate (Meso Scale Diagnostics, LLC., product number L45SA-1), and allowed to react at 37° C. for 1 hour while stirring at 700 rpm. Then, the plate was washed twice with 200 μL of 1×PBS-TP.

### (4-1) Composition of Blocking Solution

One hundred and thirty-seven (137) mM Sodium Chloride, 8.1 mM Disodium Phosphate, 2.68 mM Potassium Chloride, 1.47 mM Potassium Dihydrogenphosphate, 0.02% Tween 20, 1.5 ppm ProClin 300, and 3% BSA (bovine serum albumin)

### (4-2) Composition of 1×PBS-TP

One hundred and thirty-seven (137) mM Sodium Chloride, 8.1 mM Disodium Phosphate, 2.68 mM Potassium Chloride, 1.47 mM Potassium Dihydrogenphosphate, 0.02% Tween 20, and 1.5 ppm ProClin 300

### (5) Immobilization of Capture Probe to Measurement Plate

The capture probe was diluted 500-fold with 1×PBS-TP, and the resultant was dispensed in aliquots of 50 μL to each well of the measurement plate after the blocking. The resultant was allowed to react at 25° C. for 1 hour while stirring at 700 rpm, and then washed twice with 200 μL of 1×PBS-TP.

### (6) Immobilization of Capture Probe-Target Nucleic Acid-Assist Probe to Measurement Plate

A hybridization solution was dispensed in aliquots of 30 μL to each well of the measurement plate, and then 20 μL of the target nucleic acid was mixed therewith. The resultant was allowed to react at 25° C. for 2 hours while stirring at 700 rpm, and then washed twice with 200 μL of 1×PBS-TP.

### (6-1) Composition of Hybridization Solution

Five hundred (500) mM Tris-HCl (pH 8.0) to which 0.004 pmol/μL assist probe, 2.5M TMAC, 7×Denhardt's Solution, 0.8% N-Laurylsarcosine Sodium Salt, and 40 mM EDTA were added.

### (7) Detection Assisting Reaction

Fifty (50) μL of a detection assisting reaction solution was added to the measurement plate after immobilization of the capture probe-target nucleic acid-assist probe, and allowed to react at 25° C. for 1 hour while stirring at 700 rpm. Then, the plate was washed twice with 200 μL of 1×PBS-TP. Further, a Ru complex-labeled anti-digoxigenin antibody was prepared to 0.04 μg/mL with 1% BSA-added 1×PBS-TP, and dispensed in aliquots of 50 μL to each well. The resultant was allowed to react at 25° C. for 0.5 hours while stirring at 700 rpm, and then washed twice with 200 μL of 1×PBS-TP.

### (7-1) Composition of Detection Assisting Reaction Solution

To 280 μL of a solution (500 mM Tris-HCl (pH 7.5), 0.08% N-Laurylsarcosine Sodium Salt, and 40 mM EDTA), 525 μL of 5M TMAC, 4.9 μL of 20 pmol/μL Ru complex-labeled HCP-1, 6.1 μL of 20 pmol/μL Ru complex-labeled HCP-2, and 934.0 μL of Nuclease-Free Water were added.

### (7-2) Nucleotide Sequence of HCP-1

The nucleic acid probe (HCP-1) used in Example 4 includes a sequence complementary to a nucleotide sequence of HCP-2 in the pair of self-assembly probes, and is labeled with digoxigenin at the 5′ end.

<Nucleotide sequence of HCP-1>
5′- CAACAATCAGGACGATACCGATGAAGGATATAAGGAGTG -3′

### (7-3) Nucleotide Sequence of HCP-2

The nucleic acid probe (HCP-2) used in Example 4 includes a sequence partially complementary to the nucleotide sequence of AP-XYX-GTI2040-10 in the pair of self-assembly probes, and is labeled with digoxigenin at the 5′ end.

<Nucleotide sequence of HCP-2>
5′- GTCCTGATTGTTGCTTCATCGGTATCCACTCCTTATATC -3′

### (8) Detection

To the measurement plate after the detection assisting reaction, 150 μL of 0.5×Read Buffer [two-fold dilution of [MSD GOLD Read Buffer A (Meso Scale Diagnostics, LLC., product number R92TG-1)] with sterile purified water] was added, and light emission amount from the Ru complex-labeled anti-digoxigenin antibody was measured using a Meso QuickPlex SQ 120 MM system (manufactured by Meso Scale Diagnostics, LLC.) to detect the target nucleic acid.

### [Method of Present Invention]

### (1) Formation of AP-HCPs Assembly

A detection assisting reaction solution with the following composition was dispensed to a DNA LoBind Tube and allowed to react at 40° C. for 1 hour while stirring at 700 rpm to form an AP-HCPs assembly.

### (1-1) Composition of Detection Assisting Reaction Solution

To 160 μL of a solution (189.3 mM Tris-HCl (pH 7.5), 2.4×PBS [328.8 mM Sodium Chloride, 19.44 mM Disodium Phosphate, 6.432 mM Potassium Chloride, and 3.528 mM Potassium Dihydrogenphosphate], 3.6 mM EDTA (pH 8.0), and 0.12% Tween 20), 300 μL of 5M TMAC, 80.0 μL of 50×Denhardt's Solution, 2.5 μL of 1 pmol/μL AP-XYX-GTI2040-10, 2.8 μL of 20 pmol/μL digoxigenin-labeled HCP-1, 3.5 μL of 20 pmol/μL digoxigenin-labeled HCP-2, and 453.7 μL of Nuclease-Free Water were added.

### (2) Blocking of Measurement Plate

It was performed in the same manner as described in “(4) Blocking of measurement plate” of [Conventional method].

### (3) Immobilization of Capture Probe to Measurement Plate

It was performed in the same manner as described in “(5) Immobilization of capture probe to measurement plate” of [Conventional method].

### (4) Immobilization of Capture Probe-Target Nucleic Acid to Measurement Plate

A hybridization solution was dispensed in aliquots of 30 μL to each well of the measurement plate, and then 20 μL of the target nucleic acid was mixed therewith. The resultant was allowed to react at 25° C. for 1 hour while stirring at 700 rpm, and then washed twice with 200 μL of 1×PBS-TP.

### (4-1) Composition of Hybridization Solution

Five hundred (500) mM Tris-HCl (pH 8.0) to which 2.5 M TMAC, 7×Denhardt's Solution, 0.8% N-Laurylsarcosine Sodium Salt, and 40 mM EDTA were added.

### (5) Formation of [Capture Probe]-[Target Nucleic Acid]-[AP-HCPs Assembly] Complex

Fifty (50) μL of the AP-HCPs assembly solution was added to the measurement plate after immobilization of the capture probe-target nucleic acid, and allowed to react at 25° C. for 1 hour while stirring at 700 rpm. Then, the plate was washed twice with 200 μL of 1×PBS-TP. Further, a Ru complex-labeled anti-digoxigenin antibody was prepared to 0.04 μg/mL with 1% BSA-added 1×PBS-TP, and dispensed in aliquots of 50 μL to each well. The resultant was allowed to react at 25° C. for 0.5 hours while stirring at 700 rpm, and then washed twice with 200 μL of 1×PBS-TP.

### (6) Detection

Signal detection was performed in the same manner as described in “(8) Detection” of [Conventional method] using the washed measurement plate.

### 2. Result

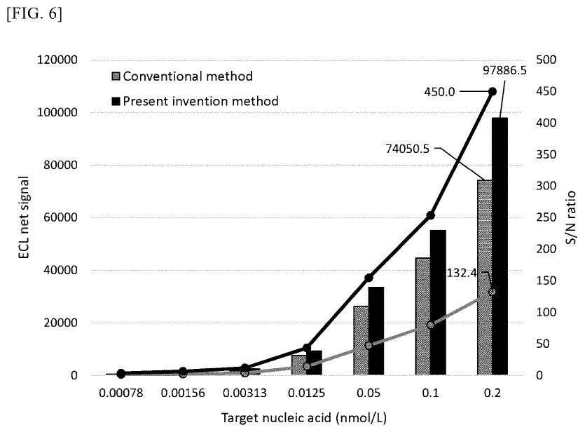

The measurement results are shown in FIG. 6. When 0.00078 to 0.2 nmol/L of the target nucleic acid was measured, the method of the present invention achieved improvement in the net signal obtained by subtracting the signal of 0 ng/mL of the blank sample by 1.2 to 1.5 times (1.32 times for 0.2 nmol/L of the target nucleic acid), and the S/N ratio by 2.1 to 3.4 times (3.4 times for 0.2 nmol/L of the target nucleic acid) as compared with the conventional method.

### INDUSTRIAL APPLICABILITY

The detection method of the present invention can be used simply and inexpensively for detecting an analyte contained in a biologically derived sample, and for designing and producing a reagent or kit for performing said detection method.

1. A method for detecting an analyte in a sample, the method comprising:
(i) providing an assembly in which a pair of self-assembly probes composed of first and second oligonucleotides and an assist probe are hybridized to each other (hereinafter, also referred to as a “signal probe polymer”);
(ii) bringing the sample containing the analyte into contact with a moiety contained in a capture probe to which the analyte binds to form a [capture probe]-[analyte] complex in a liquid phase different from that of the step (i);
(iii) bringing the signal probe polymer into contact with the [capture probe]-[analyte] complex to form a [capture probe]-[analyte]-[signal probe polymer] complex; and
(iv) detecting the [capture probe]-[analyte]-[signal probe polymer] complex.

2. A method for detecting an analyte in a sample, the method comprising:
(i) providing an assembly in which a pair of self-assembly probes composed of first and second oligonucleotides and an assist probe are hybridized to each other;
(ii) bringing the sample containing the analyte and a capture probe into contact with the assembly to form a complex of the capture probe, the analyte, the assist probe, and a plurality of first and second oligonucleotides;
(iii) removing a liquid phase from a solid phase that is bound to the capture probe or washing the solid phase, thereby removing the first and second oligonucleotides that are not involved in formation of the complex; and
(iv) detecting a label contained in the first or second oligonucleotide.

3. A method for quantifying an analyte in a sample, the method comprising:
(i) providing an assembly in which a pair of self-assembly probes composed of first and second oligonucleotides and an assist probe are hybridized to each other;
(ii) bringing the sample and a capture probe into contact with the assembly;
(iii) removing a liquid phase from a solid phase or washing the solid phase, wherein the solid phase is bound to the capture probe; and
(iv) quantifying a signal from a label contained in the first or second oligonucleotide.

4. The method according to claim 1, comprising separating and removing the analyte which is in a state free from the [capture probe]-[analyte] complex without being involved in formation of the complex.

5. The method according to claim 1, wherein the capture probe is immobilized on a solid phase before the capture probe is brought into contact with the analyte.

6. The method according to claim 1, wherein the first and second oligonucleotides are labeled with a ruthenium complex, peroxidase, fluorescent dye, biotin, or digoxigenin.

7. The method according to claim 1, wherein the sample is derived from a biological sample.

8-12. (canceled)

13. The method according to claim 2, wherein the capture probe is immobilized on a solid phase before the capture probe is brought into contact with the analyte.

14. The method according to claim 3, wherein the capture probe is immobilized on a solid phase before the capture probe is brought into contact with the analyte.

15. The method according to claim 4, wherein the capture probe is immobilized on a solid phase before the capture probe is brought into contact with the analyte.

16. The method according to claim 2, wherein the first and second oligonucleotides are labeled with a ruthenium complex, peroxidase, fluorescent dye, biotin, or digoxigenin.

17. The method according to claim 3, wherein the first and second oligonucleotides are labeled with a ruthenium complex, peroxidase, fluorescent dye, biotin, or digoxigenin.

18. The method according to claim 4, wherein the first and second oligonucleotides are labeled with a ruthenium complex, peroxidase, fluorescent dye, biotin, or digoxigenin.

19. The method according to claim 2, wherein the sample is derived from a biological sample.

20. The method according to claim 3, wherein the sample is derived from a biological sample.

21. The method according to claim 4, wherein the sample is derived from a biological sample.
